(19) United States
(12) Patent Application Publication
(10) Pub. No.: US 2017/0280972 A1
(43) Pub. Date: Oct. 5, 2017

(54) SET OF CONNECTORS, FLANGE, MANUFACTURING METHOD OF SET OF CONNECTORS, AND ENDOSCOPE

(71) Applicant: OLYMPUS CORPORATION, Tokyo (JP)

(72) Inventors: Masahiro YOSHINO, Sagamihara-shi (JP); Hiroyoshi YAJIMA, Sagamihara-shi (JP)

(73) Assignee: OLYMPUS CORPORATION, Tokyo (JP)

(21) Appl. No.: 15/623,497

(22) Filed: Jun. 15, 2017

Related U.S. Application Data
(63) Continuation of application No. PCT/JP2015/078013, filed on Oct. 2, 2015.

(30) Foreign Application Priority Data
Dec. 15, 2014 (JP) 2014-253278

(51) Int. Cl. A61B 1/00

(52) U.S. Cl. CPC A61B 1/0011; A61B 1/00112; A61B 1/00124; A61B 1/042

(57) ABSTRACT

A manufacturing method of a set of connectors includes: a step of measuring an emitting direction of light emitted from a plurality of ferrules; a step of grouping the plurality of ferrules into a first group or a second group, based on whether the emitting direction is right or left of a straight line connecting a reference fitting portion closest to the emitting direction of the light and an optical axis; a step of fabricating a plug using the ferrule belonging to the first group; and a step of fabricating a receptacle using the ferrule belonging to the second group.

### CROSS REFERENCE TO RELATED APPLICATION

This application is a continuation application of PCT/JP2015/078013 filed on Oct. 2, 2015 and claims benefit of Japanese Application No. 2014-253278 filed in Japan on Dec. 15, 2014, the entire contents of which are incorporated herein by this reference.

### BACKGROUND OF THE INVENTION

### 1. Field of the Invention

The present invention relates to a set of connectors formed of a plug and a receptacle each including an optical fiber, a flange of either the plug or the receptacle, a manufacturing method of the set of connectors, and an endoscope provided with the plug of the set of connectors.

### 2. Description of the Related Art

In an endoscope disclosed in Japanese Patent Application Laid-Open Publication No. 2014-81484, by an optical fiber scanning device (irradiation portion) arranged at a distal end portion of an insertion portion two-dimensionally scanning a distal end portion of an optical fiber that guides a laser beam from a light source device, scan irradiation of a light spot is performed.

In the endoscope system, the laser beam generated by a light source of the light source device is made incident on the optical fiber of a receptacle disposed on an outer surface of the light source device. When a plug of the endoscope is engaged with the receptacle of the light source device, the laser beam is made incident on the optical fiber of the plug, and guided to the distal end portion of the endoscope.

Japanese Patent Application Laid-Open Publication No. 2004-205604 discloses that, in a set of connectors each including a collimator, the connectors are fixed at positions of a rotation angle that minimizes coupling loss.

### SUMMARY OF THE INVENTION

A set of connectors in an embodiment of the present invention includes a first connector including a first exterior portion with an engagement portion, and a second connector including a second exterior portion with an engagement target portion to be engaged with the engagement portion. In the set of connectors, each of the first connector and the second connector includes: an optical fiber; a holding portion configured to hold the optical fiber so as to include the optical fiber; and a flange including a plurality of fitting portions formed at rotationally symmetric positions to be fitted with each of a plurality of fitting target portions provided on an inner periphery of the first exterior portion or the second exterior portion, and provided on an outer periphery of the holding portion. A fitting portion closest to an emitting direction of light emitted through the optical fiber among the plurality of fitting portions is defined as a reference fitting portion, and the first connector and the second connector are divided, based on whether the emitting direction is right or left of a straight line connecting the reference fitting portion and an optical axis.

In addition, a manufacturing method of a set of connectors in a different embodiment is a manufacturing method of a set of connectors including a first connector including a first exterior portion with an engagement portion, and a second connector including a second exterior portion with an engagement target portion to be engaged with the engagement portion, and includes: a step of inserting an optical fiber to respective through-holes of a plurality of holding portions each including a flange with a plurality of fitting portions formed on an outer periphery; a step of specifying an emitting direction of light emitted from each of the plurality of holding portions; a step of grouping the plurality of holding portions into a first group or a second group, based on whether the emitting direction is right or left of a straight line connecting a reference fitting portion closest to the emitting direction of the light among the plurality of fitting portions fitted with each of a plurality of fitting target portions provided on an inner periphery of the first exterior portion and the second exterior portion and an optical axis of the holding portion; a step of fabricating the first connector by fitting the reference fitting portion of one holding portion among the plurality of holding portions belonging to the first group with the fitting target portion at an arbitrary first relative position to the engagement portion of the first exterior portion; and a step of fabricating the second connector by fitting the reference fitting portion of one holding portion among the plurality of holding portions belonging to the second group with the fitting target portion at the first relative position to the engagement target portion of the second exterior portion.

Furthermore, for an endoscope in a different embodiment, a plug that is a first connector of a set of connectors including the first connector and a second connector is disposed at a proximal end portion of a universal cord, the second connector is a receptacle disposed at a light source device, and the set of connectors includes the first connector including a first exterior portion with an engagement portion, and the second connector including a second exterior portion with an engagement target portion to be engaged with the engagement portion. Each of the first connector and the second connector includes an optical fiber, a holding portion configured to hold the optical fiber so as to include the optical fiber, and a flange including a plurality of fitting portions formed at rotationally symmetric positions to be fitted with each of a plurality of fitting target portions provided on an inner periphery of the first exterior portion or the second exterior portion, and provided on an outer periphery of the holding portion. A fitting portion closest to an emitting direction of light emitted through the optical fiber among the plurality of fitting portions is defined as a reference fitting portion, and the first connector and the second connector are divided, based on whether the emitting direction is right or left of a straight line connecting the reference fitting portion and an optical axis.

In addition, a flange in a different embodiment is the flange provided on an outer periphery of a holding portion configured to hold an optical fiber so as to include the optical fiber, and including a plurality of fitting portions formed to be fitted with each of a plurality of fitting target portions provided on an inner periphery of an exterior portion of a connector with an engagement portion. A fitting portion closest to an emitting direction of emitted light among the plurality of fitting portions is defined as a reference fitting portion, and a part of the reference fitting portion is different in at least one of a color and a shape from other parts, based on whether the emitting direction is right or left of a straight line connecting the reference fitting portion and an optical axis.

### BRIEF DESCRIPTION OF THE DRAWINGS

FIG. 1 is a perspective view of an endoscope system including an endoscope of an embodiment;

FIG. 2 is a configuration diagram of the endoscope system including the endoscope of the embodiment;

FIG. 3 is a perspective view of a plug of a set of connectors of the embodiment;

FIG. 4 is a perspective view of a receptacle of a set of connectors of the embodiment;

FIG. 5 is an exploded view of a plug of a set of connectors of a first embodiment;

FIG. 6 is a flowchart for describing a manufacturing method of a set of connectors of the first embodiment;

FIG. 7 is a perspective view for describing the manufacturing method of a set of connectors of the first embodiment;

FIG. 8A is a projection view for describing the manufacturing method of a set of connectors of the first embodiment;

FIG. 8B is a projection view for describing the manufacturing method of a set of connectors of the first embodiment;

FIG. 9 is an exploded view for describing a set of connectors of the first embodiment;

FIG. 10 is a schematic diagram for describing an effect of a set of connectors of the first embodiment;

FIG. 11 is an exploded view for describing a set of connectors of a modification 1 of the first embodiment;

FIG. 12 is an exploded view for describing a set of connectors of a modification 2 of the first embodiment;

FIG. 13A is a projection view for describing a plug of a set of connectors of a second embodiment; and

FIG. 13B is a projection view for describing a receptacle of a set of connectors of a second embodiment.

### DETAILED DESCRIPTION OF THE PREFERRED EMBODIMENTS

Hereinafter, a set of connectors 30 of the embodiment of the present invention, a manufacturing method of the set of connectors 30, and an endoscope 90 will be described. Note that, in following descriptions, the drawings based on the individual embodiments are schematic, it should be noted that a relation of a height, a width and a depth of respective parts and a ratio of thicknesses of the respective parts or the like are different from actual ones, and even between the drawings, a part where the relation of mutual dimensions and the ratio are different is sometimes included.

### Endoscope System

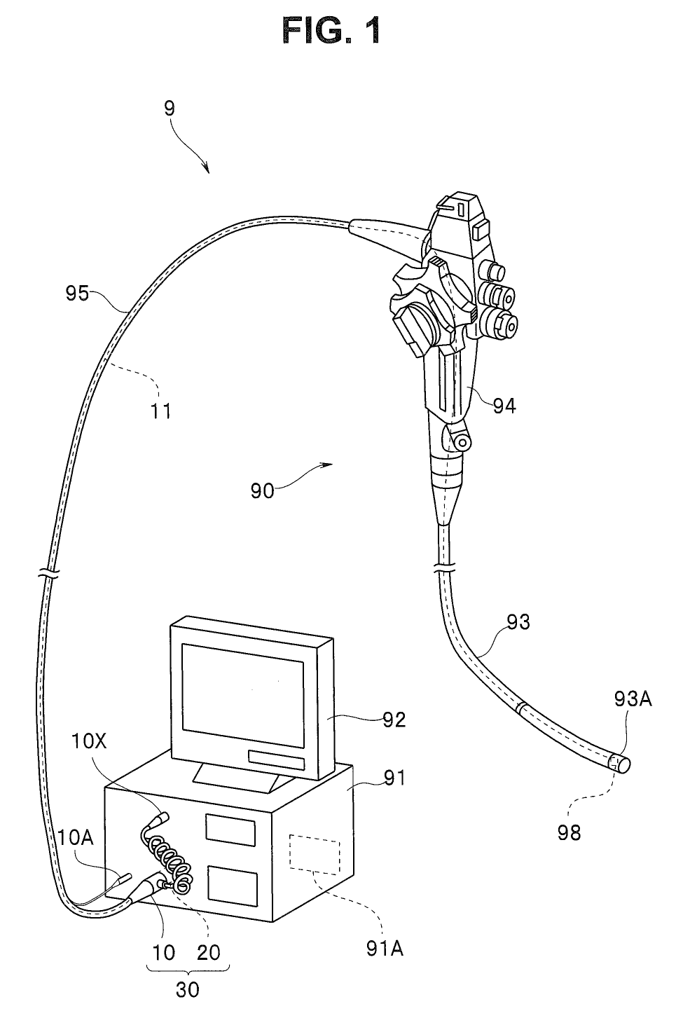

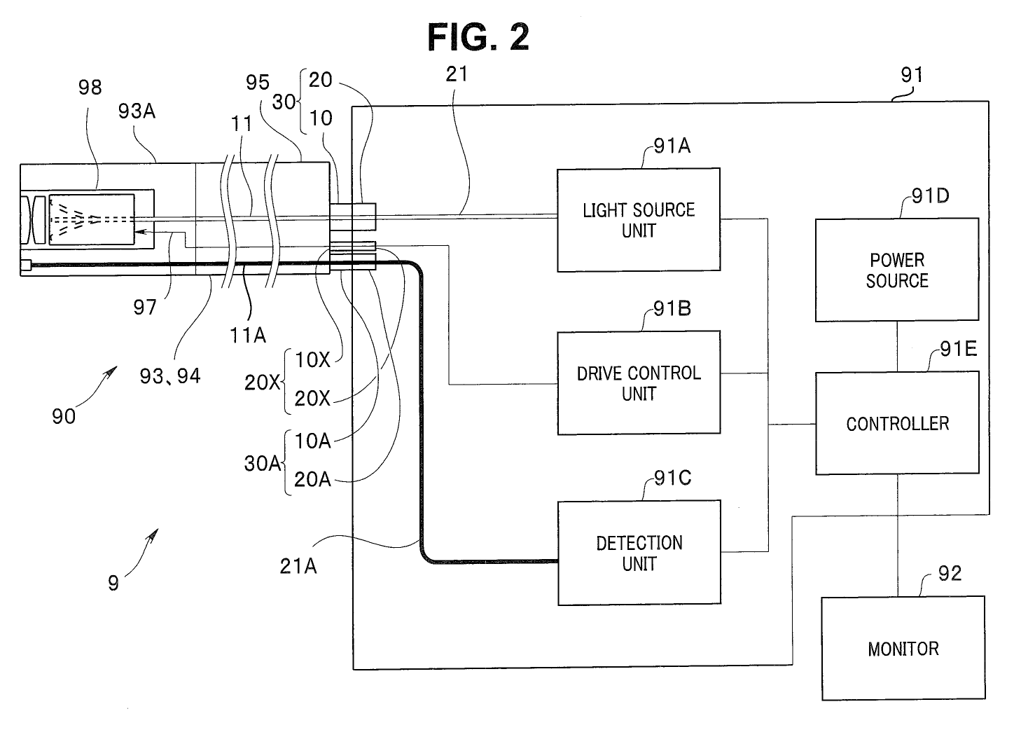

As illustrated in FIG. 1 and FIG. 2, a set of connectors 30 including a plug 10 and a receptacle 20 is a component of an endoscope system 9. FIG. 1 is a perspective view of the endoscope system, and FIG. 2 is a configuration diagram of the endoscope system.

The endoscope system 9 includes an endoscope 90, a body portion 91 having functions of a light source device and a processor, and a monitor 92. The endoscope 90 is an optical scanning type endoscope including an elongated insertion portion 93 to be inserted into a living body, a control portion 94, and a universal cable 95 (universal cord). At a proximal end portion of the universal cable 95, plugs 10, 10A and 10X are disposed. As described later, the plugs 10 and 10A are optical plugs for guiding light, and the plug 10X is an electric plug for transmitting electric signals. Then, at the body portion 91, the receptacle 20 and the like with which the plugs 10, 10A and 10X are freely attachably and detachably engaged respectively are disposed.

In the optical scanning type endoscope, a laser beam generated by a light source unit 91A of the body portion 91 is guided to an optical fiber scanning device 98 disposed at a distal end portion 93A of the insertion portion 93. The guided laser beam is two-dimensionally scanned by the optical fiber scanning device 98 and radiated to a subject. Reflected light (return light) from the subject is guided from the distal end portion 93A to the body portion 91, data processing is performed in the body portion 91, and a subject image is generated. The generated subject image is displayed on the monitor 92.

An optical fiber 11 for illumination that guides the laser beam to the optical fiber scanning device 98 is inserted from the plug 10 to the universal cable 95, the control portion 94 and the insertion portion 93 and extended to the distal end portion 93A. Note that one optical fiber 11 may be configured by bonding end portions of a plurality of optical fibers. As described later though not illustrated in FIG. 1, an optical fiber 11A of the plug 10A and a signal line 97 of the plug 10X are also inserted to the insertion portion 93 and the like and extended to the distal end portion 93A.

Note that the endoscope 90 is a so-called flexible endoscope, however, as the endoscope of the present invention, the endoscope may be a so-called rigid endoscope, the insertion portion 93 of which is rigid.

As illustrated in FIG. 2, the body portion 91 includes the light source unit 91A, a drive control unit 91B, a detection unit 91C, a power source 91D, and a controller 91E. The power source 91D supplies power to the light source unit 91A and the like. The controller 91E performs entire control of the body portion 91, and also has the function of the processor that processes signals.

The light source unit 91A generates the laser beam and emits the laser beam to an optical fiber 21. The optical fiber 21 is extended to the receptacle 20. The laser beam is guided to the optical fiber scanning device 98 at the distal end portion 93A through the optical fiber 11 of the plug 10 engaged with the receptacle 20.

Drive signals generated by the drive control unit 91B are transmitted to the optical fiber scanning device 98 at the distal end portion 93A through the plug 10X engaged with a receptacle 20X.

For example, the optical fiber scanning device 98 includes a magnetic field generation portion (not shown in the figure). At the distal end portion of the optical fiber 11 which is a light guide member for guiding the light, a permanent magnet is disposed. According to a change of strength and a direction of a magnetic field generated by the magnetic field generation portion, the distal end portion of the optical fiber 11 is two-dimensionally scanned. Note that the optical fiber scanning device 98 may be a piezoelectric drive type or the like in which the distal end portion of the optical fiber 11 is two-dimensionally scanned according to displacement of a piezoelectric body.

On the other hand, the reflected light received at the distal end portion of the optical fiber 11A arranged at the distal end portion 93A is guided to the plug 10A at the proximal end portion. Then, the light is guided to the detection unit 91C through an optical fiber 21A of a receptacle 20A engaged with the plug 10A.

As described above, the universal cable 95 is connected with the body portion 91 through connectors 30, 30A and 30X. The connectors 30, 30A and 30X are a set of connectors, each of which includes the plug 10, 10A or 10X and the receptacle 20, 20A or 20X (see FIG. 2).

### Set of Connectors

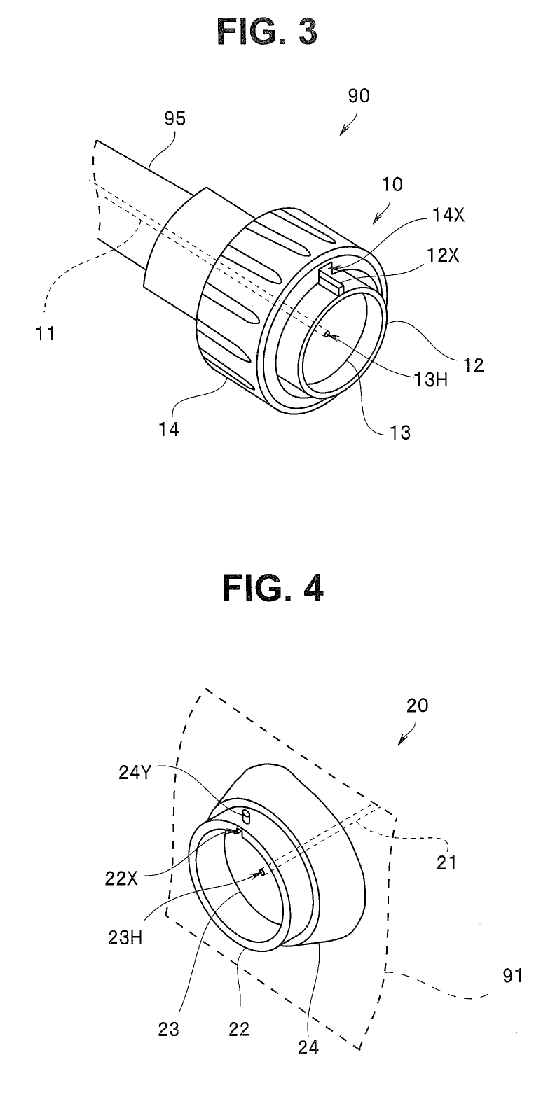

Hereinafter, the set of connectors 30 including the plug 10 that is a first connector and the receptacle 20 that is a second connector of the present embodiment will be described in detail using FIG. 3 to FIG. 5.

As illustrated in FIG. 3, the plug 10 includes a cylindrical first exterior portion 12 with a turnable ring handle 14 disposed on an outer periphery, and a ferrule 13 that is a holding portion disposed inside the first exterior portion 12. To a through-hole 13H of the ferrule 13, the optical fiber 11 is inserted and fixed.

On the other hand, inside a conical portion 24 of the receptacle 20, a cylindrical second exterior portion 22 is disposed. Then, a ferrule 23 that is a holding portion is disposed inside the second exterior portion 22. To a through-hole 23H of the ferrule 23, the optical fiber 21 is inserted and fixed.

Here, an outer diameter of the first exterior portion 12 is slightly smaller than an inner diameter of the second exterior portion 22. On the other hand, an inner diameter of the ring handle 14 is slightly larger than an outer diameter of the second exterior portion 22. Therefore, when the first exterior portion 12 is inserted to the second exterior portion 22, the plug 10 and the receptacle 20 are engaged.

A guide key 12X that is an engagement portion of the first exterior portion 12 is engaged with a key groove 22X that is an engagement target portion of the second exterior portion 22. By the guide key 12X and the key groove 22X, a relative rotation angle when the plug 10 and the receptacle 20 are engaged is uniquely stipulated. Note that the engagement portion of the first exterior portion 12 may be a recessed portion such as a key groove and the engagement target portion of the second exterior portion 22 may be a protruding portion such as a guide key.

When the plug 10 and the receptacle 20 are engaged, an engagement pin 24Y on an outer surface of the second exterior portion 22 of the receptacle 20 is inserted to a groove 14A of the ring handle 14 of the plug 10. The groove 14A is spirally extended along an inner surface of the ring handle 14. Therefore, when the ring handle 14 is turned, the inserted engagement pin 24Y is pressurized to a wall surface of the groove 14A so that the plug 10 is stably fixed to the receptacle 20. When the ring handle 14 is turned in an opposite direction, the plug 10 can be removed from the receptacle 20.

Note that the plug 10 and the receptacle 20 are greatly different externally, but internal configurations are almost same. For example, configuration of inner peripheral portions of the first exterior portion 12 and the second exterior portion 22, configurations of the ferrule 13 and the ferrule 23, and the optical fiber 11 and the optical fiber 21 or the like are same. Therefore, the configuration of the first exterior portion 12 will be described hereinafter. In addition, in the set of connectors 30 of the present embodiment, the light is guided from the receptacle 20 to the plug 10, however, for convenience of the description, an expression is sometimes given such that the light is guided from the plug 10 to the receptacle 20. Note that, hereinafter, the descriptions and illustrations or the like of the ring handle 14 or the like are omitted.

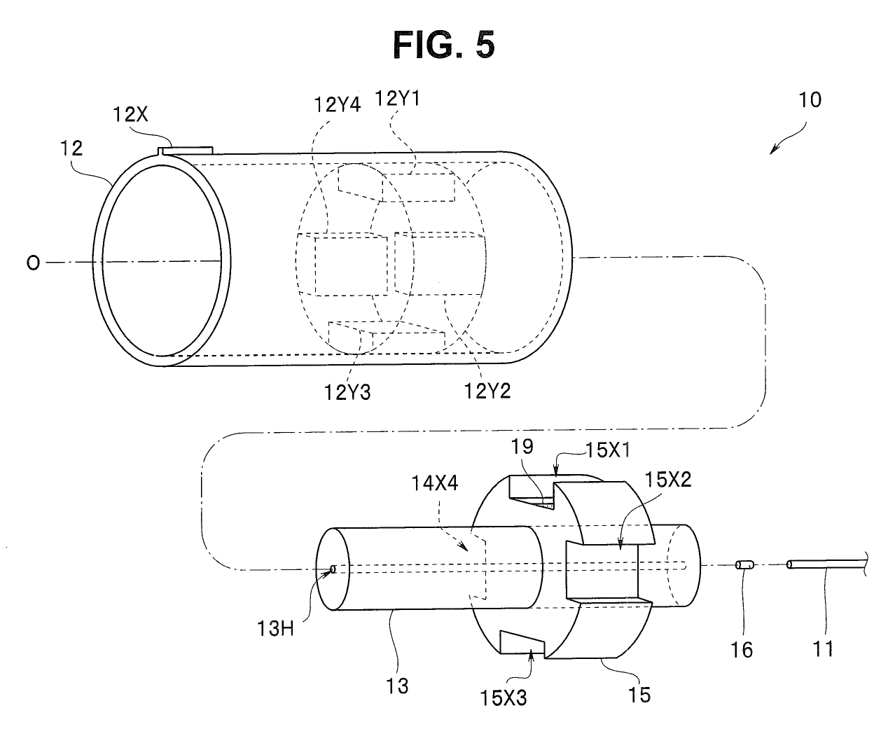

As illustrated in FIG. 5, the plug 10 includes the first exterior portion 12, the ferrule 13 that is a first holding portion, a flange 15 disposed on an outer periphery of the ferrule 13, and a collimator 16 and the optical fiber 11 inserted to the through-hole 13H of the ferrule 13. For example, the flange 15 formed of a metal such as stainless steel is attached to an outer peripheral surface of the ferrule 13 formed of zirconia or the like so that the relative rotation angle of both is fixed. Hereinafter, the flanged ferrule is simply referred to as the ferrule. Note that, in FIG. 5, a dashed line indicates an optical axis O.

An inner diameter of the through-hole 13H of the ferrule 13 is slightly larger than an outer diameter of the collimator 16 and the optical fiber 11. The collimator 16 is an optical component configured to communicate light emitted from an end face or light made incident from the end face as a parallel pencil of rays.

At the flange 15, four fitting portions 15X1-15X4 are formed at rotationally symmetric positions at every 90 degrees. Note that, hereinafter, when referring to each of a plurality of components of a same function, a numeral of one digit at an end is omitted. For example, each of the four fitting portions 15X1-15X4 is referred to as a fitting portion 15X.

On the other hand, on an inner periphery of the first exterior portion 12, four fitting target portions 12Y1-12Y4 to be fitted with each fitting portion 15X are provided. Note that relative positions of the guide key 12X formed on an outer periphery of the first exterior portion 12 and the four fitting target portions 12Y1-12Y4 formed on the inner periphery are fixed. In the plug 10, the fitting portion 15X is a recessed portion (a notched portion, a groove portion) and the fitting target portion 12Y is a protruding portion, however, the fitting portion 15X may be the protruding portion and the fitting target portion 12Y may be the recessed portion.

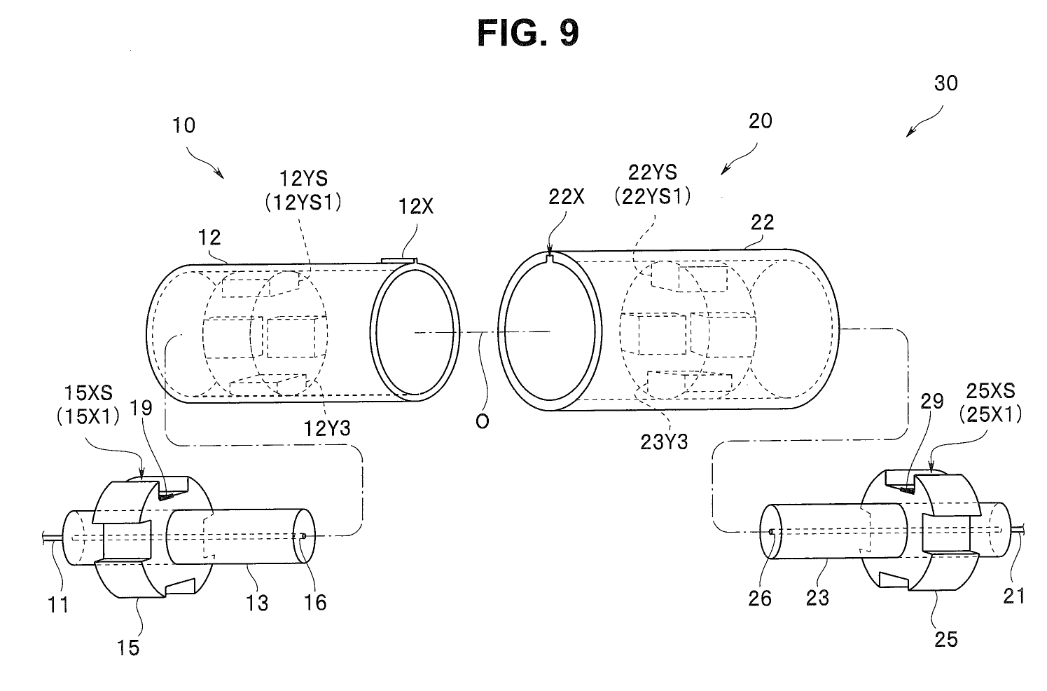

The relative positions of the key groove 22X and the four fitting target portions, both of which are formed on an inner periphery of the second exterior portion 22 are also fixed (see FIG. 9).

Note that a mark 19 is attached to a part of the fitting portion 15X1 of the flange 15 of the plug 10, and a color of a surface is different from the color of the other area. In addition, a mark 29 is also attached to one fitting portion 25X of a flange 25 of the receptacle 20 (see FIG. 9). The marks 19 and 29 will be described later in detail.

### Manufacturing Method of Set of Connectors

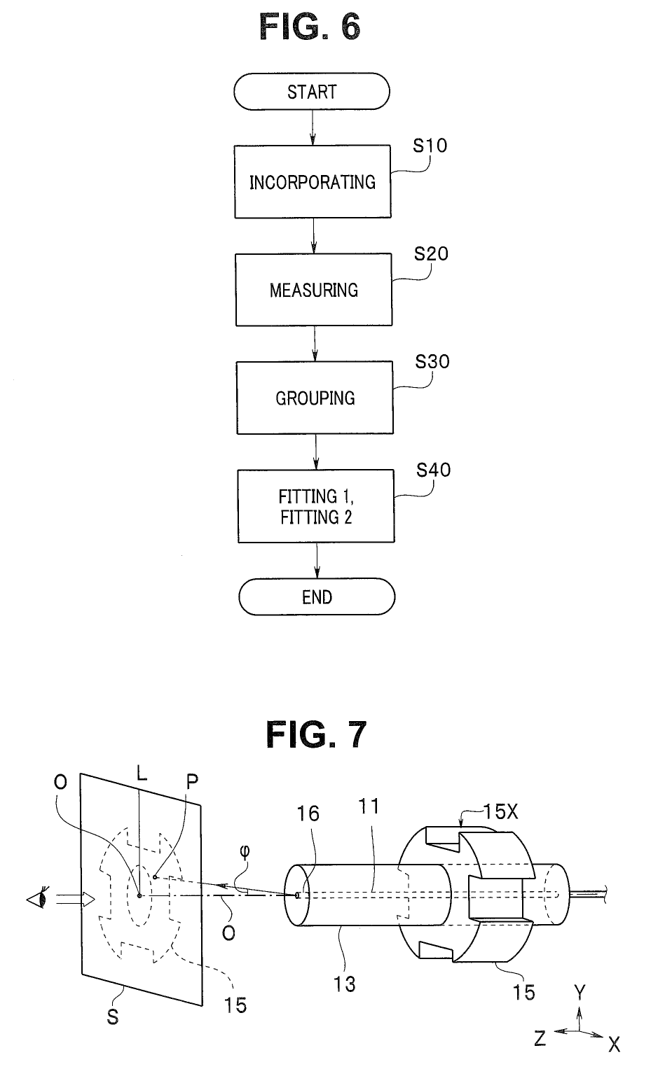

Next, along a flowchart in FIG. 6, the manufacturing method of the set of connectors 30 of the embodiment will be described.

### <Step S10> Incorporating Step

As illustrated in FIG. 5, to the through-hole 13H of the ferrule (flanged ferrule) 13 with the flange 15 disposed on the outer periphery, a gradient index lens (GRIN lens) that is the collimator 16 and the optical fiber 11 are inserted and fixed. That is, the ferrule 13 holds the optical fiber 11 and the collimator 16 so as to include the optical fiber 11 and the collimator 16.

While the collimator 16 may be configured by a plurality of lenses, for diameter reduction, it is preferable that the lenses are the gradient index lenses (GRIN lenses), a refractive index of which is non-uniform. The optical fiber 11 is a single mode optical fiber that guides the laser beam.

Note that, in a case that an outer diameter of the collimator 16 is larger than the inner diameter of the through-hole 13H, the collimator 16 may be arranged in an inside on a distal end portion side of the ferrule 13 such that the light emitted by the optical fiber 11 is made incident.

In addition, for the convenience, the ferrule or the like is described using a sign of a component of the plug 10. However, in the manufacturing method of the present embodiment, in a stage of step S10 in which the collimator and the optical fiber are incorporated in the ferrule, whether the ferrule is to be used as the plug 10 or used as the receptacle 20 is not determined.

### <Step S20> Measuring Step

For an optical connector, it is preferable that an emitting direction (incidence direction) of the light completely coincides with an optical axis direction. However, because of technical limitations, a direction of the light emitted from the ferrule 13 is not parallel to an optical axis O and is slightly inclined.

As illustrated in FIG. 7, in the measuring step, from a proximal end portion side of the optical fiber 11 of the ferrule 13 where the collimator 16 and the optical fiber 11 are held so as to be included, the light is made incident from the light source device for evaluation (not shown in the figure), and the emitting direction of the light emitted through the collimator 16 is measured.

The emitting direction of the light can be evaluated by a position of a projection point P of the light emitted from the ferrule 13, which is projected on a projection surface S vertical to the optical axis O of the ferrule 13. From a length between the projection point P and the optical axis O on the projection surface S and a distance from the ferrule 13 to the projection surface S, an inclination angle φ to the optical axis O of the emitting direction is calculated. Note that, since emission light projected to the projection surface S actually becomes a wide circle, a center point of the circle is defined as the projection point P.

In the case that the inclination angle φ of the emission light exceeds a predetermined value, for example, in the case of φ>0.3 degree, the inclination angle of the ferrule 13 may be adjusted.

Coupling loss of the engaged plug 10 and receptacle 20 is greatly different depending not only on the inclination angle φ but also the emitting direction, that is, a relative position to the optical axis O of the projection point P. When the emitting direction of the plug and the emitting direction of the receptacle 20 engaged facing each other are the same direction, the coupling loss becomes small.

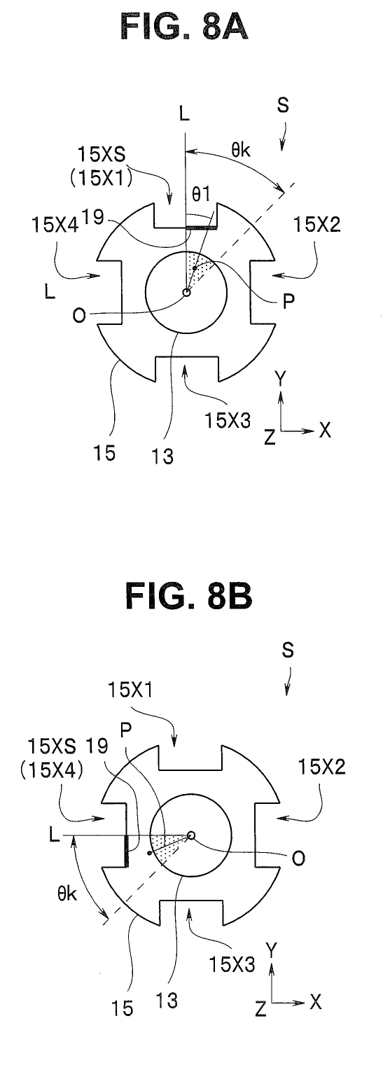

For example, in the ferrule 13 illustrated in FIG. 8A, the emitting direction (projection point P) is an upper right direction to the optical axis O. In addition, in the ferrule 13 illustrated in FIG. 8B, the emitting direction (projection point P) is a lower left direction to the optical axis O. FIG. 8A and FIG. 8B are diagrams when the projection surface S orthogonal to the emitting direction is observed from a side opposite to the ferrule 13, as illustrated in FIG. 7.

Note that a straight line L is a straight line connecting a projection position of a reference fitting portion 15XS closest to the projection point P among the four fitting portions 15X of the flange 15 projected to the projection surface S and an intersection of the optical axis O and the projection surface S, and is a reference axis of a circular coordinate system centering on the optical axis O, for stipulating the emitting direction. Note that, for a deflection angle θ of the circular coordinate system, an angle of the straight line L that is the reference axis is 0 degree, and the angle is assumed to increase clockwise.

### <Step S30> Grouping Step

First, the reference fitting portion 15XS closest to the emitting direction of the light among the four fitting portions 15X1-15X4 of the flange 15 is selected. In the ferrule 13 illustrated in FIG. 8A, the fitting portion 15X1 becomes the reference fitting portion 15XS. In the ferrule 13 illustrated in FIG. 8B, the fitting portion 15X4 becomes the reference fitting portion 15XS.

That is, among the fitting portions 15X1-15X4 projected to the projection surface S, the fitting portion at a shortest distance from the projection point P becomes the reference fitting portion 15XS.

Next, whether the projection point P indicating the emitting direction is right or left of the straight line L is determined. The emitting direction is right in the ferrule 13 illustrated in FIG. 8A, and the emitting direction is left in the ferrule 13 illustrated in FIG. 8B.

Then, based on whether the emitting direction is right or left, the plurality of holding portions are grouped into a first group or a second group. In other words, grouping is performed based on whether a deflection angle θ1 of the projection point P is positive or negative in the circular coordinate system in which the optical axis O is a center and the straight line L is the reference axis.

For example, the ferrule of the left emitting direction is grouped into the first group, and the ferrule of the right emitting direction is grouped into the second group. Note that the ferrule of the right emitting direction may be grouped into the first group, and the ferrule of the left emitting direction may be grouped into the second group conversely.

Here, for the emitting direction (projection point) of the light of the ferrule belonging to the first group, the deflection angle θ is in a range (θk=45 degrees) of 0 degree to 45 degrees in the circular coordinate system. On the other hand, for the emitting direction of the light of the ferrule belonging to the second group, the deflection angle θ is in the range (θk=45 degrees) of −45 degrees to 0 degree (315 degrees to 360 degrees).

Note that it is preferable to attach the mark 19 (see FIG. 8A or the like) and change the color so as to identify which fitting portion is the reference fitting portion 15XS among the four fitting portions 15X.

Note that marking is not necessary in the case that the plurality of grouped ferrules are temporarily kept until a next fitting step in a storage case or the like in which each reference fitting portion 15XS is arranged at a predetermined position.

### <Step S40> Fitting Step

The plurality of grouped ferrules are respectively fitted with the first exterior portion 12 or the second exterior portion 22 to fabricate the connector.

For example, the ferrule 13 belonging to the first group is fitted with the first exterior portion 12 and turned to the plug 10 which is the first connector. In contrast, the ferrule 23 belonging to the second group is fitted with the second exterior portion 22 and turned to the receptacle 20 which is the second connector.

As illustrated in FIG. 5, the four fitting target portions 12Y to be fitted with the four fitting portions 15X of the flanged ferrule 13 are provided on the inner surface of the first exterior portion 12. Therefore, the first exterior portion 12 and the ferrule 13 can be fitted at four relative rotation angles. In addition, four fitting target portions 22Y to be fitted with the four fitting portions 25X of the flanged ferrule 23 are provided on the inner surface of the second exterior portion 22. Therefore, the second exterior portion 22 and the ferrule 23 can be fitted at four relative rotation angles. Each fitting target portion 12Y is at a predetermined relative position with the guide key 12X. Furthermore, each fitting target portion 22Y is at a predetermined relative position with the key groove 22X.

### Fitting Step 1

As illustrated in FIG. 9, by fitting the reference fitting portion 15XS of one ferrule 13 among the plurality of ferrules belonging to the first group with a reference fitting target portion 12YS, the plug 10 which is the first connector is fabricated. The reference fitting target portion 12YS is the fitting target portion at an arbitrary first relative position to the guide key 12X of the first exterior portion 12.

Here, the reference fitting target portion 12YS may be any one of the four fitting target portions 12Y1-12Y4. In FIG. 9, the fitting target portion 12Y1 is the reference fitting target portion 12YS.

### Fitting Step 2

Next, by fitting a reference fitting portion 25XS (25X1) of one ferrule 23 among the plurality of ferrules belonging to the second group with a reference fitting target portion 22YS at the first relative position to the key groove 22X of the second exterior portion 22, the receptacle 20 which is the second connector is fabricated. That is, for the receptacle 20, the ferrule 23 with the flange 25 into which a collimator 26 and the optical fiber 21 are inserted is fitted with the second exterior portion 22.

Here, the first relative position is the relative position of the guide key 12X and the fitting target portion 12Y1. That is, among the four fitting target portions 12Y, the fitting target portion 12Y1 is at a closest position right below the guide key 12X in a planar view from the optical axis direction. Therefore, a fitting target portion 22Y1 at the closest position right below the key groove 22X in the planar view from the optical axis direction becomes the reference fitting target portion 22YS.

Note that, in the case that the reference fitting portion 15XS of the ferrule 13 is fitted with a fitting target portion 12Y3, a fitting target portion 22Y3 becomes the reference fitting target portion.

For the emitting direction of the light of the ferrule 13 of the plug 10 illustrated in FIG. 8A, the deflection angle θ is in the range (θk=45 degrees) of 0 degree to 45 degrees in the circular coordinate system in which the optical axis O is the center, the straight line L is the reference axis and the deflection angle θ increases clockwise. On the other hand, for the emitting direction of the light of the ferrule 23 of the receptacle 20 illustrated in FIG. 8B, the deflection angle θ is in the range (θk=45 degrees) of −45 degrees to 0 degree (315 degrees to 360 degrees). Then, the plug 10 and the receptacle 20 are engaged so as to face each other. Note that the collimator 16 of the engaged plug 10 and the collimator 26 of the receptacle 20 are arranged through a predetermined gap without being in contact.

As already described, in the endoscope system, the light source device and the endoscope are not always used in a determined combination. Therefore, there is a risk that the coupling loss becomes different depending on the combination of the light source device and the endoscope and a light quantity of illumination light changes. When the light quantity of the illumination light is small, an image becomes dark and visibility declines. Furthermore, when the light quantity is larger than specifications, the image is too bright and the visibility also declines.

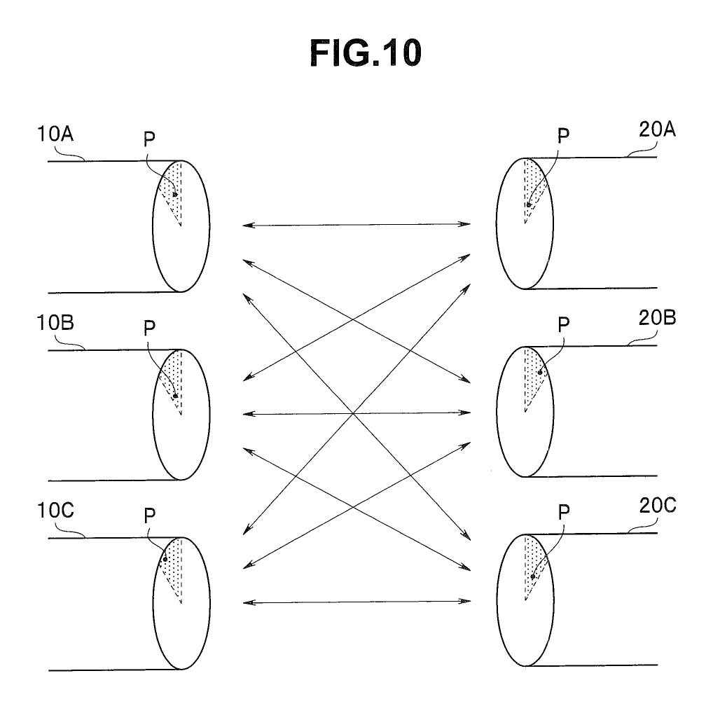

However, as illustrated in FIG. 10, the plurality of plugs 10 (10A, 10B, 10C) and the plurality of receptacles 20 (20A, 20B, 20C) are engaged such that “the range of the deflection angle 45 degrees) including the emitting direction of the light faces each other even when arbitrarily engaged.

Therefore, in the set of connectors 30 including the plug 10 and the receptacle 20, even when an engaging opponent is different, the coupling loss does not greatly decline and predetermined coupling loss is obtained. In addition, in the endoscope 90 including the plug 10 of the set of connectors 30, the predetermined coupling loss is obtained even when the connected light source unit 91A is different.

### Modifications

Next, sets of connectors 30D and 30E or the like of modifications 1 and 2 of the first embodiment will be described. Since the sets of connectors 30D and 30E or the like are similar to the set of connectors 30 of the first embodiment or the like and have the same effect, same signs are attached to components of the same function and the descriptions are omitted.

In the sets of connectors 30D and 30E of the modifications, the reference fitting portion corresponding to the emitting direction of the light is different in the shape from the other fitting portions. Then, the reference fitting target portions of the first exterior portion and the second exterior portion are fitted only with the reference fitting portion among the plurality of fitting portions.

### Modification 1

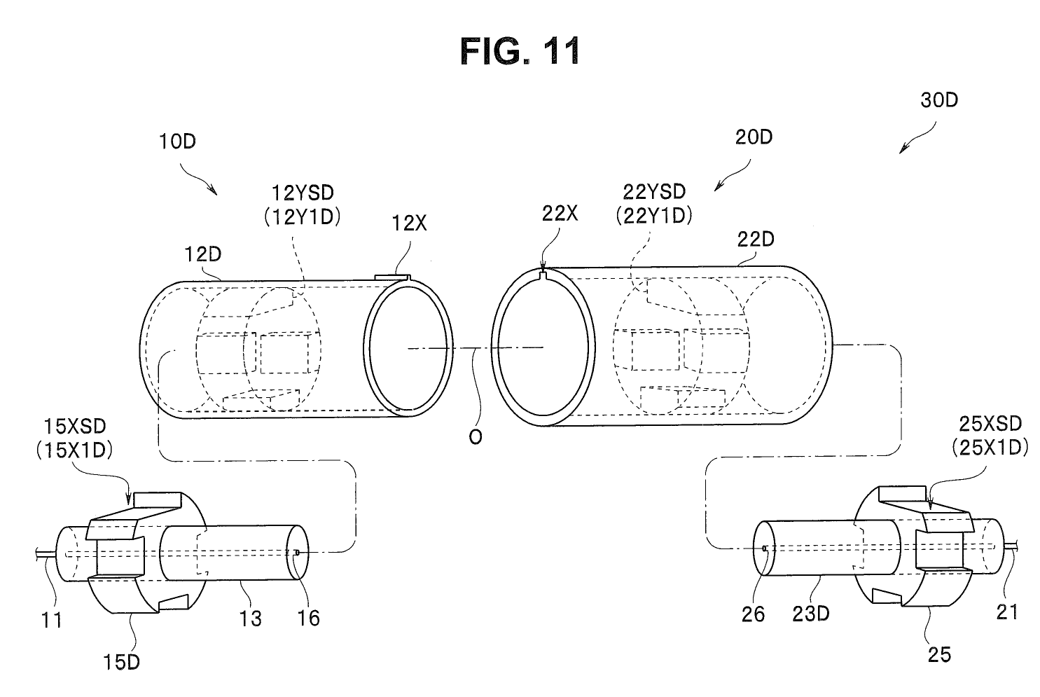

As illustrated in FIG. 11, the set of connectors 30D of the modification 1 includes a plug 10D that is the first connector and a receptacle 20D that is the second connector. The plug 10D is disposed at the proximal end portion of the endoscope 90, and the receptacle 20D is disposed on the outer surface of the light source unit 91A (body portion 91).

For a reference fitting portion 15XSD (15X1D) of a ferrule 13D of the plug 10D, differently from the other fitting portions 15X, an area corresponding to the light emitting direction is cut off. That is, in a step (S20) of measuring the emitting direction of the light, in order to identify the measured emitting direction, a part of a flange 15D is cut off by cutting or abrasive machining Note that, while the reference fitting portion of the ferrule 13D can be identified from the other fitting portions since the shape is different, identification may be further facilitated by attaching a mark or changing the color.

Then, for a first exterior portion 12D, a fitting target portion 12Y1D which is the reference fitting target portion is different in the shape from the other fitting target portions. Therefore, the reference fitting portion 15XSD (15X1D) of the plug 10D can be fitted only with the fitting target portion 12Y1D of the first exterior portion 12D. In other words, in the first exterior portion 12D, a reference fitting target portion 12YSD is predetermined.

Similarly, a reference fitting portion 25XSD (25X1D) of a ferrule 23D of the receptacle 20D can be fitted only with a fitting target portion 22Y1D (22YSD) of a second exterior portion 22D.

### Modification 2

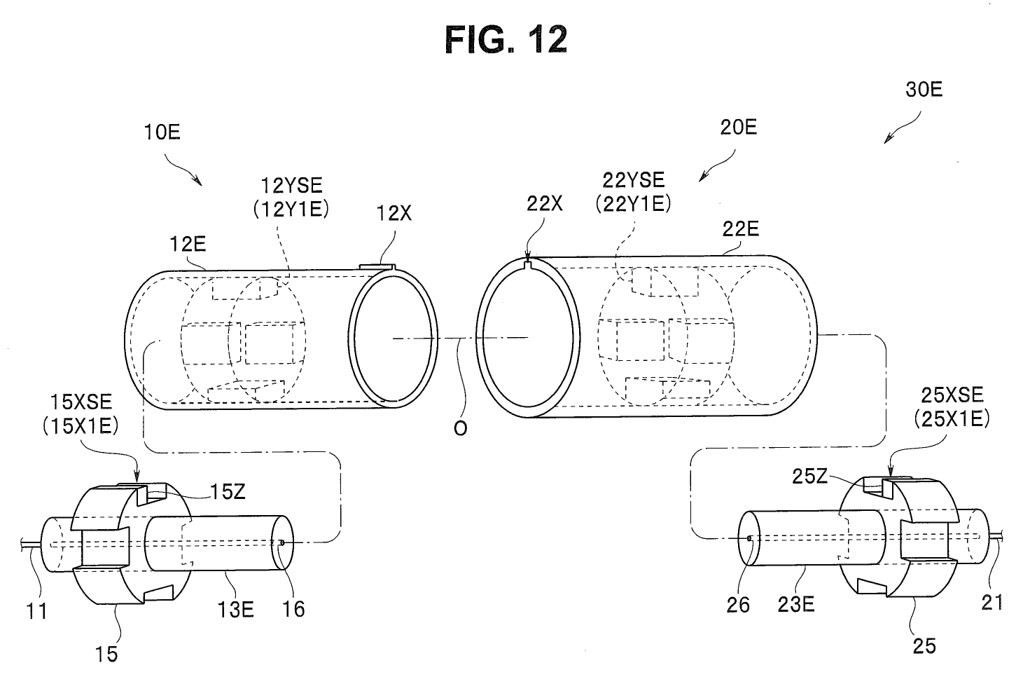

As illustrated in FIG. 12, the set of connectors 30E of the modification 2 includes a plug 10E that is the first connector and a receptacle 20E that is the second connector. The plug 10E is disposed at the proximal end portion of the endoscope 90, and the receptacle 20E is disposed on the outer surface of the light source unit 91A (body portion 91).

For a reference fitting portion 15XSE (15X1E) of the plug 10E, differently from the other fitting portions 15X, a member 15Z is disposed at a recessed portion corresponding to the light emitting direction. That is, in the step (S20) of measuring the emitting direction of the light, in order to identify the measured emitting direction, the member 15Z is attached to the fitting portion 15X1E.

Then, for a first exterior portion 12E, a fitting target portion 12Y1E is different from the other fitting target portions. Therefore, the reference fitting portion 15XSE (15X1E) of the plug 10E can be fitted only with the fitting target portion 12Y1E of the first exterior portion 12E. In other words, in the first exterior portion 12E, the reference fitting target portion is predetermined.

Similarly, since a member 25Z is arranged, a reference fitting portion 25XSE (25X1E) of the receptacle 20E can be fitted only with a fitting target portion 22Y1E which is the reference fitting target portion 22YSE of a second exterior portion 22E.

Note that, in the sets of connectors 30D and 30E of the modifications 1 and 2, only one of the plug and the receptacle may be the configuration of the modification and the other may be the configuration of the embodiment 1, or one may be the configuration of the modification 1 and the other may be the configuration of the modification 2.

### Second Embodiment

Next, a set of connectors 30F or the like of the second embodiment will be described. The set of connectors 30F or the like is similar to the set of connectors 30 of the first embodiment or the like and has the same effect, the same signs are attached to the components of the same function and the descriptions are omitted.

In the first embodiment, at the flange 15 (25) of the ferrule 13 (23), four recessed portions 15X1-15X4 (25X1-25X4) are formed as fitting portions at the rotationally symmetric positions.

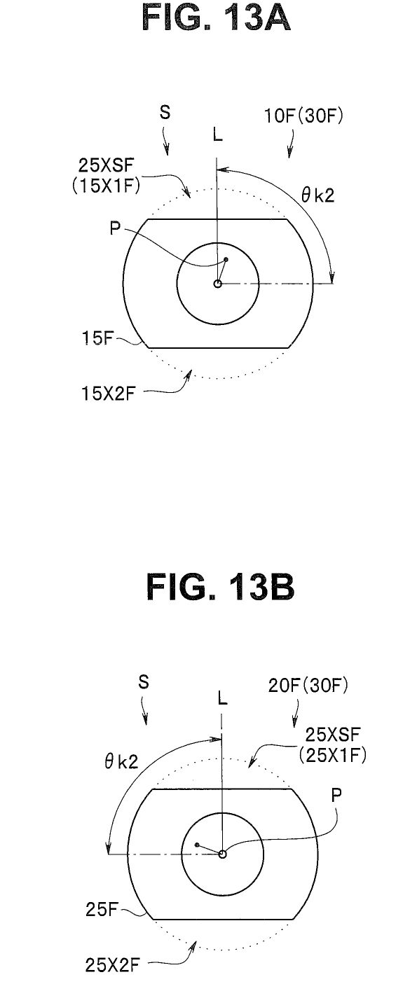

In contrast, as illustrated in FIG. 13A, in a plug 10F of the set of connectors 30F of the second embodiment, two recessed portions 15X1F and 15X2F are formed as the fitting portions at the rotationally symmetric positions at a flange 15F. Then, as illustrated in FIG. 13B, in a receptacle 20F of the set of connectors 30F of the second embodiment, two recessed portions 25X1F and 25X2F are formed as the fitting portions at the rotationally symmetric positions at a flange 25F.

Note that, though not illustrated, at the first exterior portion fitted with the plug 10F and the second exterior portion fitted with the receptacle 20F, corresponding fitting target portions are respectively formed.

Also in the manufacturing method of the present embodiment, the emitting direction of the light is measured by the measuring step similar to the manufacturing method of the first embodiment, and the plurality of ferrules are grouped into the first group or the second group by the grouping step.

However, in the plug 10F, for the emitting direction (projection point) of the light of the ferrule belonging to the first group, the deflection angle θ is in a range (θk=90 degrees) of 0 degree to 90 degrees in the circular coordinate system. On the other hand, for the emitting direction of the light of the ferrule of the receptacle 20F belonging to the second group, the deflection angle θ is in the range (θk=90 degrees) of −90 degrees to 0 degree (270 degrees to 360 degrees).

Then, in the set of connectors 30F, the plug 10F and the receptacle 20F are engaged so as to face each other.

Therefore, as illustrated in FIG. 10, the plurality of plugs 10F and the plurality of receptacles 20F are engaged such that “the range of the deflection angle 90 degrees” including the emitting direction of the light faces each other even when arbitrarily engaged.

For the set of connectors 30F, the effect is not great compared to the set of connectors 30 or the like, however, even when the engaging opponent is different, the coupling loss does not greatly decline and the predetermined coupling loss is obtained.

As described above, in the case that the number of the plurality of fitting portions formed at the flange is N, Ok becomes 180/N (degrees). That is, since it is N=4 in the set of connectors 30 of the first embodiment, it is θk=45 degrees. In contrast, since it is N=2 in the set of connectors 30F of the second embodiment, it is θk=90 degrees.

When the number N of the plurality of fitting portions is larger, the risk that the coupling loss declines is prevented more even when the engaging opponent is different. When the number N of the fitting portions is 2 or larger, the predetermined effect is obtained. Note that an upper limit of the number N of the fitting portions is 16 for example regarding machining accuracy or the like.

The present invention is not limited to the embodiments or the modifications or the like described above, and can be variously changed or modified or the like without changing the gist of the present invention.

What is claimed is:

1. A set of connectors comprising
a first connector including a first exterior portion with an engagement portion, and a second connector including a second exterior portion with an engagement target portion to be engaged with the engagement portion,
wherein each of the first connector and the second connector includes:
an optical fiber;
a holding portion configured to hold the optical fiber so as to include the optical fiber; and
a flange including a plurality of fitting portions formed at rotationally symmetric positions to be fitted with each of a plurality of fitting target portions provided on an inner periphery of the first exterior portion or the second exterior portion, and provided on an outer periphery of the holding portion, wherein
a fitting portion closest to an emitting direction of light emitted through the optical fiber among the plurality of fitting portions is defined as a reference fitting portion, and the first connector and the second connector are divided, based on whether the emitting direction is right or left of a straight line connecting the reference fitting portion and an optical axis.

2. The set of connectors according to claim 1, further comprising
a collimator configured to communicate light with the optical fiber and emit the light,
wherein the collimator is held to be included by the holding portion.

3. The set of connectors according to claim 1, wherein a part of the reference fitting portion is different in at least one of a color and a shape from other parts.

4. The set of connectors according to claim 1, wherein the first connector is a plug disposed at a proximal end portion of a universal cord of an endoscope, and the second connector is a receptacle disposed at a light source device.

5. The set of connectors according to claim 2, wherein the collimator is a GRIN lens, a refractive index of which is non-uniform.

6. A manufacturing method of a set of connectors including a first connector including a first exterior portion with an engagement portion, and a second connector including a second exterior portion with an engagement target portion to be engaged with the engagement portion, comprising:
a step of inserting an optical fiber to respective through-holes of a plurality of holding portions each including a flange with a plurality of fitting portions formed on an outer periphery;
a step of specifying an emitting direction of light emitted from each of the plurality of holding portions;
a step of grouping the holding portions into a first group or a second group, based on whether the emitting direction is right or left of a straight line connecting a reference fitting portion closest to the emitting direction among the plurality of fitting portions fitted with each of a plurality of fitting target portions provided on an inner periphery of the first exterior portion and the second exterior portion and an optical axis of the holding portion;
a step of fabricating the first connector by fitting the reference fitting portion of the holding portions belonging to the first group with the fitting target portion at an arbitrary first relative position to the engagement portion of the first exterior portion; and
a step of fabricating the second connector by fitting the reference fitting portion of the holding portions belonging to the second group with the fitting target portion at the first relative position to the engagement target portion of the second exterior portion.

7. The manufacturing method of a set of connectors according to claim 6, wherein the four fitting portions are formed at the flange.

8. The manufacturing method of a set of connectors according to claim 7,
wherein, in the step of grouping,
a mark for identifying the emitting direction of the light to be emitted is attached to the flange.

9. A set of connectors manufactured by the manufacturing method according to claim 6.

10. The set of connectors according to claim 9, wherein the first connector is a plug disposed at a proximal end portion of a universal cord of an endoscope, and the second connector is a receptacle disposed at a light source device.

11. An endoscope comprising the plug according to claim 4.

12. A flange provided on an outer periphery of a holding portion configured to hold an optical fiber so as to include the optical fiber, and
including a plurality of fitting portions formed to be fitted with each of a plurality of fitting target portions provided on an inner periphery of an exterior portion of a connector with an engagement portion,
wherein a fitting portion closest to an emitting direction of emitted light among the plurality of fitting portions is defined as a reference fitting portion, and a part of the reference fitting portion is different in at least one of a color and a shape from other parts, based on whether the emitting direction is right or left of a straight line connecting the reference fitting portion and an optical axis.
